(19) United States
(12) Patent Application Publication
(10) Pub. No.: US 2014/0014641 A1
(43) Pub. Date: Jan. 16, 2014

(54) MELTABLE FUEL WARMER ASSEMBLY

(71) Applicant: Travis Propes, Charleston, SC (US)

(72) Inventor: Travis Propes, Charleston, SC (US)

(21) Appl. No.: 13/937,025

(22) Filed: Jul. 8, 2013

Related U.S. Application Data
(60) Provisional application No. 61/670,056, filed on Jul. 10, 2012.
(60) Provisional application No. 61/786,545, filed on Mar. 15, 2013.

(51) Int. Cl. A61L 9/03; H05B 3/26

(52) U.S. Cl. CPC A61L 9/03; H05B 3/26
USPC 219/421; 219/436

(57) ABSTRACT

A warmer (14) comprises a retainer support assembly (28), a heater (30), and an air mover (32). The retainer support assembly (28) is adapted to support a fuel retainer assembly (12). The heater (30) selectively heats the retainer support assembly (28). The air mover (32) is coupled to the retainer support assembly (28), and moves air substantially adjacent to the retainer support assembly (28). The retainer support assembly (28) includes a retainer supporter (54) that is adapted to retain the fuel retainer assembly (12), and a support shaft (56) that supports the retainer supporter (54). Additionally, the retainer supporter (54) can include one or more support vents (62), with activation of the air mover (32) directing air through the support vents (62). The retainer support assembly (28) can also include a second retainer supporter (254B) that is adapted to simultaneously retain a second fuel retainer assembly (212B).

### RELATED APPLICATIONS

This application is related to and claims domestic priority under 35 U.S.C. §119(e) from U.S. Provisional Application Ser. No. 61/670,056, filed Jul. 10, 2012, entitled “WAX WARMER WITH DISPOSABLE DROP-INS”, and from U.S. Provisional Application Ser. No. 61/786,545, filed Mar. 14, 2013, entitled “WAX WARMER WITH DISPOSABLE DROP-INS” As far as permitted, the contents of U.S. Provisional Application Ser. Nos. 61/670,056 and 61/786,545 are incorporated herein by reference.

### BACKGROUND

A candle warmer or a wax warmer (also referred to herein simply as a “warmer”) is an electric warmer that melts a candle or scented wax to release its scent and/or to provide a certain ambience. For example, meltable fuel, such as wax tarts, melt tarts or wax melts are placed into a warmer, which melts the wax within a body of the warmer. Unfortunately, such warmers can be difficult and cumbersome to clean, e.g., to remove the residual wax within the warmer, between uses. In addition, conventional warmers can fail to adequately disperse the scent of the wax, resulting in only a faint scent being detected by the user. Moreover, these types of warmers do not allow for changes in scents without complete removal and replacement of the wax within the warmer by the user, which can be inconvenient and disruptive in a social setting.

### SUMMARY

The present invention is directed toward a warmer for use with a fuel retainer assembly of a warmer assembly. In certain embodiments, the warmer comprises a retainer support assembly, a heater, and an air mover. The retainer support assembly is adapted to support the fuel retainer assembly. The heater selectively heats the retainer support assembly. The air mover is coupled to the retainer support assembly. Additionally, the air mover moves air substantially adjacent to the retainer support assembly.

In some embodiments, the retainer support assembly includes a retainer supporter that is adapted to retain the fuel retainer assembly. Additionally, in one such embodiment, the retainer supporter includes one or more support vents. In such embodiment, activation of the air mover directs air through the support vents. Further, in one embodiment, the retainer support assembly can further include a support shaft that supports the retainer supporter. In such embodiment, the air mover can be positioned about the support shaft.

In one embodiment, the warmer further comprises a warmer body, wherein the retainer support assembly, the air mover and the heater are positioned substantially within the warmer body.

Additionally, in one embodiment, the retainer support assembly includes a first retainer supporter that is adapted to retain the fuel retainer assembly, and a second retainer supporter that is adapted to simultaneously retain a second fuel retainer assembly.

The present invention is further directed toward a warmer assembly including a fuel retainer assembly having a meltable fuel and a fuel retainer that retains the meltable fuel, and a warmer as described above that selectively retains the fuel retainer assembly, wherein the heater selectively provides heat the retainer support assembly to melt the meltable fuel. In one embodiment, the retainer support assembly includes a retainer supporter that is adapted to retain the fuel retainer assembly, the retainer supporter having an inner support surface having a first shape. Additionally, in such embodiment, the fuel retainer has an outer surface having a second shape, the second shape being substantially similar to the first shape.

In another application, the present invention is directed toward a warmer for use with a first fuel retainer assembly and a second fuel retainer assembly, the warmer comprising (I) a retainer support assembly that is adapted to simultaneously retain the first fuel retainer assembly and the second fuel retainer assembly; and (ii) a heater assembly that selectively provides heat to the retainer support assembly.

Additionally, in another application, the present invention is directed toward a warmer for use with a first fuel retainer assembly, the warmer comprising (A) a retainer support assembly including (i) a first retainer supporter that is adapted to retain the first fuel retainer assembly, the first retainer supporter including one or more first support vents, and (ii) a support shaft that supports the first retainer supporter; (B) a first heater that selectively provides heat to the first retainer supporter; (C) an air mover that is positioned about the support shaft, wherein activation of the air mover directs air through the first support vents; (D) a warmer body, wherein the retainer support assembly, the first heater and the air mover are positioned substantially within the warmer body; and (E) a power source that selectively activates the first heater and the air mover.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1A is a simplified front view illustration of an embodiment of a warmer assembly having features of the present invention;

FIG. 1B is a simplified partially exploded front view illustration of the warmer assembly illustrated in FIG. 1A;

FIG. 1C is a simplified top view illustration of a portion of the warmer assembly illustrated in FIG. 1A;

FIG. 1D is a simplified bottom view illustration of the warmer assembly illustrated in FIG. 1A;

FIG. 2A is a simplified front view illustration of another embodiment of the warmer assembly;

FIG. 2B is a simplified partially exploded front view illustration of the warmer assembly illustrated in FIG. 2A;

FIG. 2C is a simplified top view illustration of a portion of the warmer assembly illustrated in FIG. 2A;

FIG. 3A is a simplified front view illustration of still another embodiment of the warmer assembly;

FIG. 3B is a simplified top view illustration of the warmer assembly illustrated in FIG. 3A;

FIG. 4A is a simplified front view illustration of yet another embodiment of the warmer assembly;

FIG. 4B is a simplified side view illustration of the warmer assembly illustrated in FIG. 4A;

FIG. 5A is a simplified front view of still yet another embodiment of the warmer assembly; and

FIG. 5B is a simplified top view illustration of the warmer assembly illustrated in FIG. 5A.

### DESCRIPTION

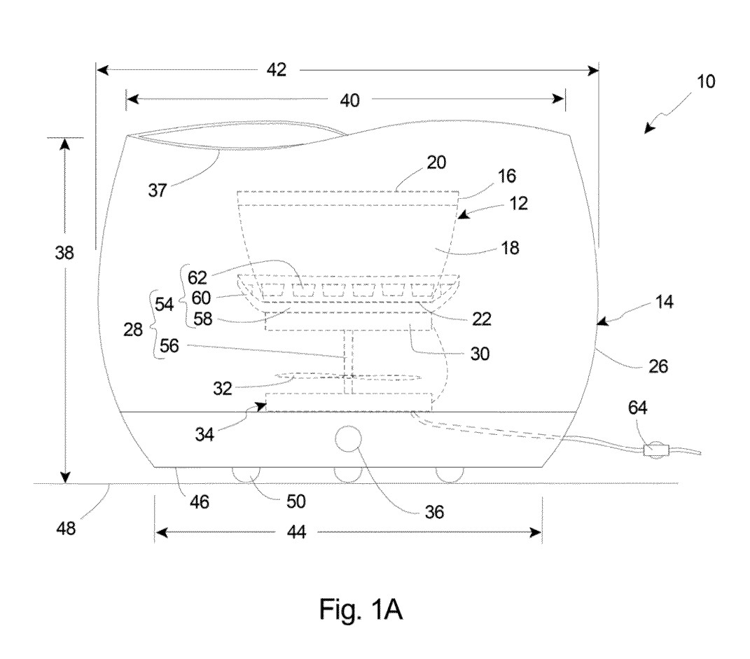

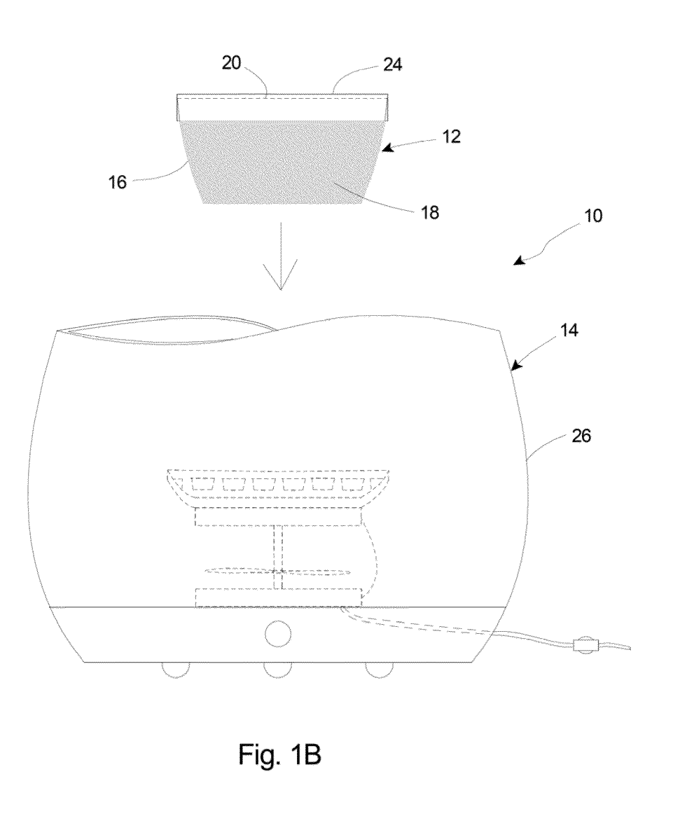

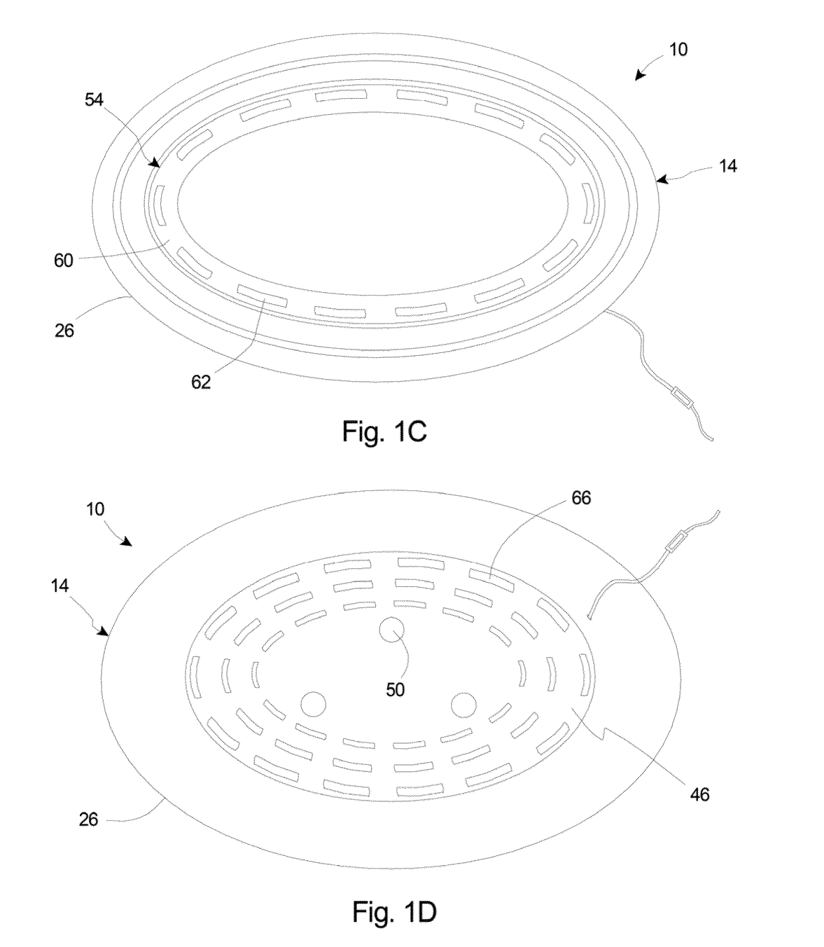

FIG. 1A is a simplified front view illustration of an embodiment of a meltable fuel warmer assembly 10 (also referred to herein simply as a “warmer assembly”) having features of the present invention. The design of the warmer assembly 10 can be varied. In the embodiment illustrated in FIG. 1A, the warmer assembly 10 includes a fuel retainer assembly 12 (illustrated in phantom), and a warmer 14 that selectively receives and retains the fuel retainer assembly 12.

As illustrated, the fuel retainer assembly 12 includes a fuel retainer 16 (illustrated in phantom) and a meltable fuel 18 (illustrated in phantom), e.g., a candle, scented wax, or other appropriate meltable fuel. The fuel retainer 16 retains or otherwise holds the meltable fuel 18. Additionally, the fuel retainer assembly 12 is selectively positioned at least substantially, if not entirely, within the warmer 14 when it is desired to use the warmer assembly 10.

In one embodiment, the fuel retainer 16 is a glass cup that can be easily positioned within and removed from the warmer 14. Alternatively, the fuel retainer 16 can have a different design. For example, the fuel retainer 16 can be made from a material other than glass, such as ceramic, metal, composite materials, etc. Additionally, in some embodiments, the fuel retainer 16 can have a cross-section that is substantially oval-shaped and the fuel retainer 16 can be tapered somewhat such that a retainer top 20 (illustrated in phantom) of the fuel retainer 16 is somewhat larger and/or wider than a retainer bottom 22 (illustrated in phantom) of the fuel retainer 16. Alternatively, the fuel retainer 16 can have a different cross-sectional shape, and/or the retainer top 20 and the retainer bottom 22 of the fuel retainer 16 can be have relative sizes that are different than those specifically illustrated. For example, in certain non-exclusive alternative embodiments, the fuel retainer 16 can have a cross-section that is substantially circle-shaped, square-shaped, rectangle-shaped, hexagon-shaped, octagon-shaped, or some other shape.

Additionally, in certain embodiments, the fuel retainer assembly 12 can be designed to be disposable such that the fuel retainer 16 and/or the meltable fuel 18 can be simply thrown away after one or more uses. For example, in one embodiment, the fuel retainer 16 can be a single-use cup that is simply thrown away after one use, thereby enabling use of the warmer assembly 10 to be more convenient and tidy. Alternatively, the fuel retainer assembly 12 can be reusable.

Further, in some embodiments, the fuel retainer 16 can include a removable lid 24 (illustrated in FIG. 1B) to better preserve the meltable fuel 18 within the fuel retainer 16 prior to use, e.g., during shipping and storage. For example, in one embodiment, the fuel retainer 16 can include a frosted acetate lid. Alternatively, the lid 24 can have a different design and/or composition, or the fuel retainer 16 can be designed without a lid 24.

In some embodiments, the fuel retainer 16 can be designed to retain between approximately 1.00 ounces and 2.50 ounces of meltable fuel 18 prior to initial use. For example, in one specific non-exclusive embodiment, the fuel retainer 16 can be designed to retain approximately 1.25 ounces of meltable fuel 18 prior to initial use. Alternatively, the fuel retainer 16 can be designed to retain greater than 2.50 ounces or less than 1.00 ounces of meltable fuel 18.

Moreover, as provided herein, the meltable fuel 18 can be a scented wax that melts when heated so as to release any scent contained within the meltable fuel 18. Alternatively, the meltable fuel 18 can have a different design. It should be noted that although only a single fuel retainer assembly 12 is shown in FIG. 1A, the warmer assembly 10 can be used with multiple retainer assemblies 12 without the need for extensive cleaning between uses of the warmer assembly 10, such as is required with a typical warmer assembly 10. Additionally, it should also be noted that the specific meltable fuel 18, e.g., the particular scents or aromas that can be provided from melting of the meltable fuel 18, can be different within each of the retainer assemblies 12 if desired.

The design of the warmer 14 can be varied to suit the specific requirements of the warmer assembly 10. In the embodiment illustrated in FIG. 1A, the warmer 14 includes a warmer body 26, a retainer support assembly 28 (illustrated in phantom), a heater 30 (illustrated in phantom), an air mover 32 (illustrated in phantom), a power source 34 (illustrated partially in phantom) and an indicator light 36. Alternatively, the warmer 14 can have a different design. For example, the warmer 14 can be designed without the air mover 32 and/or without the indicator light 36.

As an overview, the warmer 14 of the present invention provides an enhanced ability for effectively circulating and dispersing a desired scent, aroma or fragrance that emanates from the fuel retainer assembly 12 upon melting of the meltable fuel 18 within the fuel retainer 16. More particularly, the retainer support assembly 28 and the air mover 32, as described in greater detail herein below, are designed to enable the warmer 14 to provide better and more effective fragrance throw (circulation) upon the melting of the meltable fuel 18. Additionally, the warmer 14 further provides enhanced ability for mixing, combining and/or changing any desired scents, aromas or fragrances that may be produced through use of the warmer assembly 10.

The warmer body 26 selectively receives and retains the fuel retainer assembly 12. Stated in another manner, the fuel retainer assembly 12 can be selectively positioned within the warmer body 26 when it is desired to use the warmer assembly 10, i.e. when it is desired to create the desired ambience of light and/or scent that can be generated through use of the warmer assembly 10. In one embodiment, as illustrated in FIG. 1A, the fuel retainer assembly 12 is positioned substantially entirely within the warmer body 26 when it is desired to use the warmer assembly 10. Alternatively, the fuel retainer assembly 12 can be positioned within the warmer body 26 such that the fuel retainer assembly 12 extends slightly above a top edge 37 of the warmer body 26.

Additionally, as illustrated, the retainer support assembly 28, the heater 30 and the air mover 32 can all be positioned substantially within the warmer body 26.

In certain embodiments, the warmer body 26 can have the general shape of a portion of a compressed sphere. In some such embodiments, the warmer body 26 can have a height 38 of between approximately 3.0 and 6.0 inches, a top width 40 of between approximately 2.5 and 5.5 inches, a middle (maximum) width 42 of between approximately 3.5 and 6.5 inches, and a base width 44 of between approximately 2.0 and 5.0 inches. Alternatively, the warmer body 26 can have dimensions that are larger or smaller than those specifically listed above and/or the warmer body 26 can be designed to have another suitable shape.

Further, in certain embodiments, the warmer body 26 can have a base 46 that is substantially flat to enable the warmer body 26 to be positioned stably on a surface 48, such as a table top, counter top, or other suitable surface. Additionally and/or alternatively, as illustrated, the warmer body 26 can include one or more base supports 50, e.g., projections that extend in a generally downward direction away from the base 46 of the warmer body 26. In such embodiment, the base supports 50 can engage the surface 48 and maintain the base 46 of the warmer body 26 spaced apart from the surface 48 upon which the warmer 14 is placed. In the embodiment illustrated in FIG. 1A, the warmer body 26 includes three base supports 50 that are positioned substantially evenly spaced apart from one another about the base 46 to stably support the warmer 14 on the surface 48. Alternatively, the warmer body 26 can include more than three or fewer than three base supports 50. For example, in one embodiment, the warmer body 26 can be designed without any base supports 50 and the base 46 can rest directly upon the surface 48.

Additionally, as illustrated, the warmer body 26 can include the top edge 37 that is somewhat undulating around a perimeter to provide a more pleasing appearance, and/or to allow the user to more easily insert and/or remove the fuel retainer assembly 12 from the warmer body 26.

Additionally, the warmer body 26 can be made from various suitable materials and/or can be made to be any of various colors. For example, in one non-exclusive embodiment, the warmer body 26 can be made from an off-white ceramic material. Moreover, in one embodiment, a portion of the warmer body 26, e.g., near the base 46 of the warmer body 26, can made from an unfinished ceramic material, and another portion of the warmer body 26, e.g., near the top edge 37 of the warmer body 26, can be made from a glazed ceramic material. Additionally and/or alternatively, the warmer body 26 can be made from other suitable materials, such as glass, metal, composite materials, etc., and/or the warmer body 26 can have one or more different colors.

As shown in FIG. 1A, the retainer support assembly 28 can be positioned substantially, if not entirely, within the warmer body 26. Additionally, the retainer support assembly 28 receives, retains and/or supports the fuel retainer assembly 12 when the fuel retainer assembly 12 is positioned within the warmer body 26. The design and positioning of the retainer support assembly 28 can be varied depending on the specific requirements of the fuel retainer assembly 12 and/or the warmer assembly 10. In the embodiment illustrated in FIG. 1A, the retainer support assembly 28 includes a retainer supporter 54 (illustrated in phantom) and a support shaft 56 (illustrated in phantom).

The retainer supporter 54 is positioned to substantially directly receive and support the fuel retainer assembly 12 when the fuel retainer assembly 12 is positioned within the warmer body 26. Additionally, the retainer supporter 54 can be used to inhibit the meltable fuel 18 for otherwise spilling within the warmer body 26, e.g., onto the heater 30.

The design of the retainer supporter 54 can be varied. As illustrated, the retainer supporter 54 can include a substantially flat, support base 58 (illustrated in phantom in FIG. 1A) and a support wall 60 (illustrated in phantom in FIG. 1A) that extends generally upward and outward from a perimeter of the support base 58. With this design, the retainer supporter 54 can be somewhat bowl-shaped to more effectively support and retain the fuel retainer assembly 12 when the fuel retainer assembly 12 is positioned within the warmer body 26. As illustrated in FIG. 1A, the support wall 60 of the retainer supporter 54 can be angled such that at least a majority of the support wall 60 is spaced apart from the fuel retainer 16 when the fuel retainer assembly 12 is positioned within the warmer body 26. Alternatively, the support wall 60 can be angled and/or shaped such that the support wall 60 is substantially directly adjacent to or in contact with the fuel retainer 16 when the fuel retainer assembly 12 is positioned within the warmer body 26. In such alternative embodiment, the retainer supporter 54 can have a shape that substantially matches the shape of at least a bottom portion of the fuel retainer 16.

Further, as shown in FIG. 1A, the retainer supporter 54 can include one or more support vents 62 (illustrated in phantom) that are positioned spaced apart from one another on the support wall 60 and/or the support base 58 of the retainer supporter 54, e.g., at or near a perimeter of the retainer supporter 54. Additionally and/or alternatively, the retainer supporter 54 can be designed without any support vents 62 and/or the support vents 62 can be positioned about the retainer supporter 54 in a different manner. For example, one or more of the support vents 62 can be positioned within the support base 58 of the retainer supporter 54.

The support shaft 56 is coupled to and/or secured to the retainer supporter 54 and supports the retainer supporter 54 relative to the base 46 of the warmer body 26. In this embodiment, the support shaft 56 extends between the base 46 of the warmer body 26 and the retainer supporter 54. Alternatively, the support shaft 56 can support the retainer supporter 54 in a different manner and/or the support shaft 56 can be positioned at a different location within the warmer body 26. Additionally, the support shaft 56 can be substantially cylinder-shaped, or the support shaft 56 can have another suitable shape.

The heater 30 heats the retainer supporter 54, which in turn, heats the fuel retainer 18. Heating of the fuel retainer 18 causes the meltable fuel 18 to melt within the fuel retainer 16. The design and/or positioning of the heater 30 can be varied. As shown, the heater 30 can be positioned substantially, if not entirely, within the warmer body 26. In various embodiments, the heater 30 is electrically coupled to and/or positioned near, substantially adjacent to and/or within the retainer supporter 54 to provide the necessary heat to the retainer supporter 54 for melting the meltable fuel 18 within the fuel retainer 16. During use, the heater 30 can be selectively turned on and off by manipulation of the power source 34. More particularly, when the heater 30 is activated, the heater 30 provides heat to the retainer supporter 54 that can be used to warm the fuel retainer 16, and, thus, to warm and melt the meltable fuel 18 that is retained within the fuel retainer 16. Additionally, when the heater 30 is not activated, e.g., when the power source 34 is turned off, no heat is provided by the heater 30, and the fuel retainer assembly 12 can be removed from the warmer body 26 and/or one or more additional retainer assemblies 12 can be added to or exchanged within the warmer body 26. Alternatively, the fuel retainer assembly 12 can be exchanged while the heater 30 is activated, although this may not be advisable, and care should be used during this process as the fuel retainer assembly 12 can be hot.

The air mover 32 selectively circulates air within and around the warmer body 26. In one embodiment, the air mover 32 can include a fan, which is used to disperse the scent, aroma or fragrance that emanates from the fuel retainer assembly 12 upon the melting of the meltable fuel 18 when the fuel retainer assembly 12 is heated. In other embodiments, the air mover 32 can include other types of devices that are designed to circulate air. The design and/or positioning of the air mover 32 can be varied. As illustrated, the air mover 32 can be positioned substantially horizontally (i.e. relative to the surface 48) below the retainer supporter 54, substantially parallel to the retainer supporter 54, and near the base 46 of the warmer body 26. Alternatively, the air mover 32 can have a different design and/or be positioned in a different manner.

Moreover, in certain embodiments, the air mover 32 is coupled to the retainer support assembly 28. For example, in the embodiment illustrated in FIG. 1A, the air mover 32 is positioned and/or coupled about the support shaft 56. With this design, when the air mover 32 is activated by the power source 34, the air mover 32 can rotate with (e.g., the support shaft 56 itself can be rotated) and/or about the support shaft 56 to move air within the warmer body 26 in a generally upward direction toward the retainer supporter 54. Additionally, the air mover 32 can direct air through the support vents 62 that are provided within the retainer supporter 54. The support vents 62 are designed to enable better air flow, e.g., from the operation of the air mover 32, and, thus, to enable better fragrance dispersion (circulation) upon the melting of the meltable fuel 18 within the fuel retainer 16.

The air mover 32 can be of any suitable size and can have any desired number of alternative settings. For example, when activated, the air mover 32 can be selectively settable to function on high speed, medium speed, low speed, or any other desired speed. In certain alternative embodiments, the air mover 32 can include one or more discrete speed settings, or the air mover 32 can include a continuum of speed settings between a maximum speed setting and a minimum (i.e. off) speed setting.

The power source 34 selectively provides power to the heater 30 and the air mover 32. More particularly, the power source 34 can selectively activate the heater 30, e.g., between an on and off setting and/or between varying heat levels; and the power source 34 can selectively activate the air mover 32, e.g., between an on and off setting and/or between varying speed settings. Additionally, in certain embodiments, the power source 34 can include a timer mechanism that allows the heater 30 and/or the air mover 32 to be selectively activated and/or deactivated for any desired lengths of time.

As illustrated in FIG. 1A, the power source 34 can include a wired connection for plugging the warmer body 26, and, thus, the warmer 14 into an outlet (not illustrated). Further, as illustrated, the power source 34 can include a toggle switch 64 that enables different settings and/or different activations. For example, the toggle switch 64 can include modes or settings for off, on (with just the heater 30 being activated), and on (with both the heater 30 and the air mover 32 being activated). Additionally and/or alternatively, the toggle switch 64 can further include modes or settings for only activating the air mover 32, for having different speed settings for the air mover 32, as described above, and/or for having different heat level settings for the heater 30. Still alternatively, the power source 34 can have a different design. For example, the power source 34 can be designed to be directly plugged into an outlet without the need for a wire or cord, and/or the power source 34 can include one or more batteries that can be positioned and retained within the warmer body 26.

The indicator light 36 provides an indication of whether or not the warmer 14 is on. More specifically, the indicator light 36 can provide such an indication when power is being supplied to the heater 30 within the warmer body 26 and/or when the heater 30 is at least at a certain temperature. As such, the indicator light 36 can warn a user to avoiding touching any parts of the warmer 14 that may be hot or otherwise uncomfortable for the user to contact. In alternative embodiments, the indicator light 36 can be blue, red, white, orange, green, or some other color. Additionally, in the embodiment illustrated in FIG. 1A, the indicator light 36 is positioned near the base 46 of the warmer body 26. Alternatively, the indicator light 36 can be positioned at a different location on the warmer body 26.

FIG. 1B is a simplified partially exploded front view illustration of the warmer assembly 10 shown in FIG. 1A. More particularly, FIG. 1B illustrates the fuel retainer assembly 12, i.e. the fuel retainer 16 containing the meltable fuel 18, positioned above the warmer 14 and ready to be positioned within the warmer body 26. Additionally, FIG. 1B further illustrates that the fuel retainer 16 can include the lid 24 that is positioned on the retainer top 20 to better preserve the meltable fuel 18 within the fuel retainer 16 prior to use, e.g., during shipping and storage.

FIG. 1C is a simplified top view illustration of a portion of one embodiment of the warmer assembly 10, such as that shown in FIG. 1A. More particularly, FIG. 1C is a simplified top view illustration of the warmer 14, with the fuel retainer assembly 12 having been removed for purposes of clarity. As shown in FIG. 1C, the warmer body 26 can have a cross-section that is substantially oval-shaped to match the substantially oval-shaped cross-section of the fuel retainer 16 (illustrated in FIG. 1A). Additionally, the retainer supporter 54 can also be substantially oval-shaped so as to more effectively receive and retain the fuel retainer assembly 12 (illustrated in FIG. 1A). Alternatively, the warmer body 26 can have a cross-section that is other than substantially oval-shaped, and/or the retainer supporter 54 can be other than substantially oval-shaped. For example, in certain non-exclusive, alternative embodiments, the cross-section of the warmer body 26 and/or the retainer supporter 54 can be substantially circle-shaped, square-shaped, rectangle-shaped, hexagon-shaped, octagon-shaped, or some other shape.

Additionally, FIG. 1C further illustrates the one or more support vents 62 that can be incorporated into the design of the retainer supporter 54. As provided above, the one or more support vents 62 are designed to provide and/or enable better air flow, e.g., from the operation of the air mover 32 (illustrated in FIG. 1A), and, thus, better fragrance throw (circulation) upon the melting of the meltable fuel 18 (illustrated in FIG. 1A). As illustrated, in certain embodiments, the support vents 62 can be positioned spaced apart from one another about the support wall 60 of the retainer supporter 54, i.e, at or near a perimeter of the retainer supporter 54. Moreover, the support vents 62 can be positioned substantially completely about the perimeter of the retainer supporter 54. For example, in one embodiment, the retainer supporter 54 can include fourteen support vents 62 that are substantially equally spaced apart about the perimeter of the retainer supporter 54. Alternatively, the retainer supporter 54 can include greater than fourteen or fewer than fourteen support vents 62, and/or the support vents 62 can be positioned in a different manner about the retainer supporter 54.

FIG. 1D is a simplified bottom view illustration of one embodiment of the warmer assembly 10, such as that shown in FIG. 1A. In particular, FIG. 1D illustrates one potential non-exclusive design of the base 46 of the warmer body 26. As shown, the base 46 of the warmer body 26 can be substantially oval-shaped. Alternatively, the base 46 can be substantially circle-shaped, square-shaped, rectangle-shaped, hexagon-shaped, octagon-shaped, or some other shape.

Additionally, FIG. 1D further illustrates that the base 46 can include one or more base vents 66 to provide better air flow through the base 46 of the warmer body 26, i.e. between the base 46 of the warmer body 26 and the surface 48 (illustrated in FIG. 1A) upon which the warmer 14 is placed. Moreover, the base vents 66 further allow for the dissipation of any heat that is generated within the warmer body 26 during operation of the warmer 14. In various alternative embodiments, the base 46 can include any suitable number of base vents 66, and the base vents 66 can be positioned about the base 46 of the warmer body 26 in any suitable manner or pattern.

Further, FIG. 1D also illustrates the positioning of the base supports 50 that support the warmer 14 relative to the surface 48 in this particular embodiment. As illustrated, the warmer 14 can include three base supports 50 that are substantially evenly spaced apart from one another about the base 46 of the warmer body 26 in a triangular pattern. Alternatively, as noted above, the warmer body 26 can include greater than three or fewer than three base supports 50, and/or the base supports 50 can be positioned in a different manner about the base 46 of the warmer body 26.

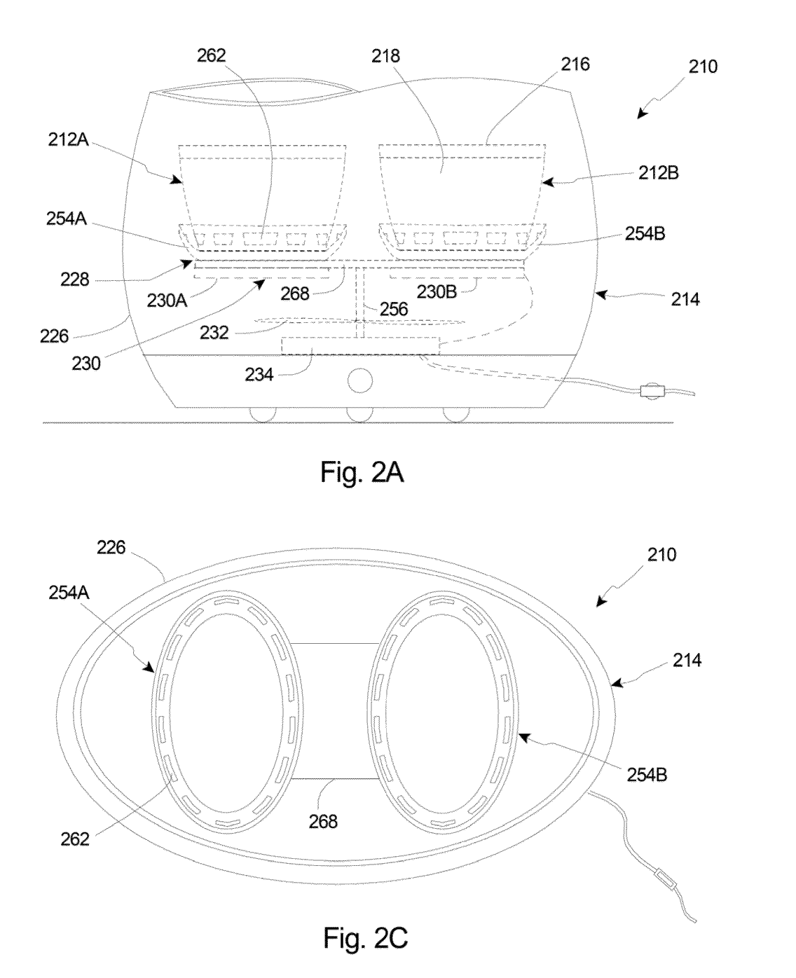

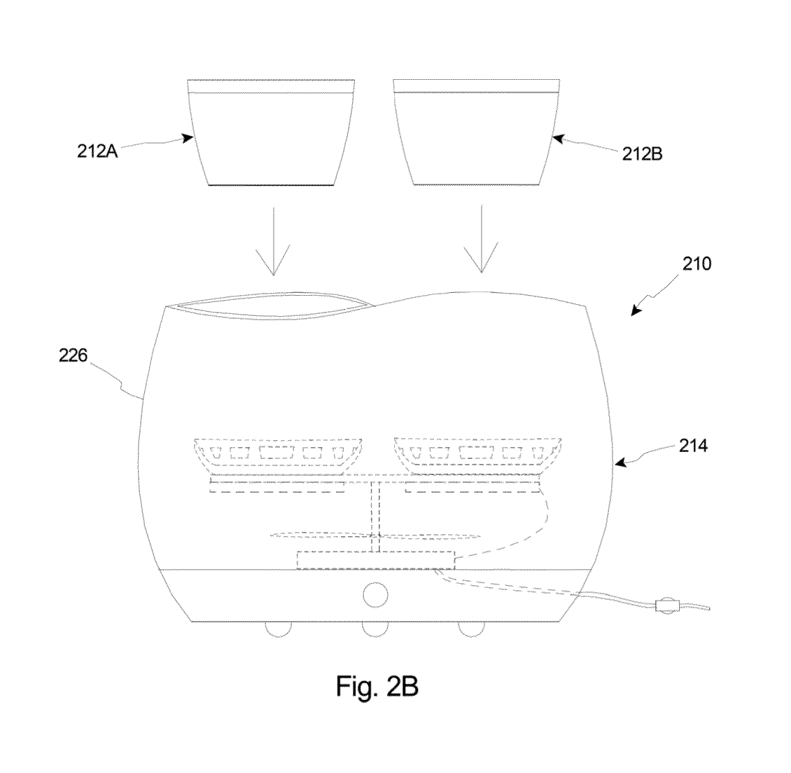

FIG. 2A is a simplified front view illustration of another embodiment of the warmer assembly 210. The design of the warmer assembly 210 is somewhat similar to the warmer assembly 10 illustrated and described above in relation to FIGS. 1A-1D. For example, the warmer assembly 210 again includes a warmer 214 that is somewhat similar to the warmer 14 illustrated and described above in relation to FIGS. 1A-1D. However, in this embodiment, the warmer 214 includes a retainer support assembly 228 (illustrated in phantom) that is designed to simultaneously receive, retain and/or support two retainer assemblies, i.e. a first fuel retainer assembly 212A (illustrated in phantom) and a second fuel retainer assembly 212B (illustrated in phantom).

Each of the first fuel retainer assembly 212A and the second fuel retainer assembly 212B can be substantially similar to the fuel retainer assembly 12 illustrated and described above. For example, as illustrated, each of the retainer assemblies 212A, 212B include a fuel retainer 216 (illustrated in phantom) and a meltable fuel 218 (illustrated in phantom), e.g., a candle, scented wax, or other appropriate meltable fuel. It should be appreciated that in different applications of the warmer assembly 210, the meltable fuel 218 utilized in the first fuel retainer assembly 212A can be substantially the same as or different from the meltable fuel 218 utilized in the second fuel retainer assembly 212B. Additionally, it should be appreciated that by utilizing different meltable fuel 218 in each of the retainer assemblies 212A, 212B, the warmer assembly 210 is better able to create various scent combinations in order to more effectively create any desired ambience.

In the embodiment illustrated in FIG. 2A, in order to effectively and simultaneously receive, retain and/or support the first fuel retainer assembly 212A and the second fuel retainer assembly 212B, the retainer support assembly 228 includes (i) a first retainer supporter 254A (illustrated in phantom), (ii) a second retainer supporter 254B (illustrated in phantom), and (iii) a plate coupler 268 (illustrated in phantom) that extends substantially between and couples the first retainer supporter 254A and the second retainer supporter 254B.

Each of the first retainer supporter 254A and the second retainer supporter 254B can be substantially similar to one another and substantially similar to the retainer supporter 54 illustrated and described above in relation to FIGS. 1A-1D. For example, each of the first retainer supporter 254A and the second retainer supporter 254B can again be somewhat bowl-shaped so as to more effectively support and retain the first fuel retainer assembly 212A and the second fuel retainer assembly 212B, respectively. Additionally, each of the retainer supporters 254A, 254B can again include one or more support vents 262 (illustrated in phantom) to enable better air flow, e.g., from the operation of the air mover 232 (illustrated in phantom), and, thus, to enable better fragrance throw (circulation) upon the melting of the meltable fuel 218 with the fuel retainer 216 in each of the retainer assemblies 212A, 212B.

As provided above, the plate coupler 268 extends substantially between and couples the first retainer supporter 254A and the second retainer supporter 254B. Additionally, as illustrated, the plate coupler 268 enables both of the retainer supporters 254A, 254B to be supported by a single support shaft 256 (illustrated in phantom). More specifically, the support shaft 256 can be coupled to and/or substantially directly support the plate coupler 268, with the plate coupler 268, in turn, providing support for the first retainer supporter 254A and the second retainer supporter 254B. Alternatively, the warmer 214 can be designed without the plate coupler 268 and/or the retainer support assembly 228 can include individual support shafts for individually supporting each of the first retainer supporter 254A and the second retainer supporter 254B.

Additionally, as illustrated in FIG. 2A, the warmer 214 can include a heater assembly 230 (illustrated in phantom) having a first heater 230A (illustrated in phantom) and a second heater 230B (illustrated in phantom). In various embodiments, the first heater 230A is electrically coupled to and/or positioned near, substantially adjacent to and/or within the first retainer supporter 254A. With this design, the first heater 230A provides heat to the first retainer supporter 254A to melt the meltable fuel 218 within the fuel retainer 216 of the first fuel retainer assembly 212A, as desired. Similarly, in various embodiments, the second heater 230B is electrically coupled to and/or positioned near, substantially adjacent to and/or within the second retainer supporter 254B. With this design, the second heater 230B provides heat to the second retainer supporter 254B to melt the meltable fuel 218 within the fuel retainer 216 of the second fuel retainer assembly 212B, as desired.

During use, the power source 234 (illustrated partially in phantom) can selectively and individually activate the first heater 230A and the second heater 230B. More particularly, when the first heater 230A is activated by the power source 234, the first heater 230A provides heat to the first retainer supporter 254A that can be used to warm and melt the meltable fuel 218 that is retained within the fuel retainer 216 of the first fuel retainer assembly 212A; and when the second heater 230B is activated by the power source 234, the second heater 230B provides heat to the second retainer supporter 254B that can be used to warm and melt the meltable fuel 218 that is retained within the fuel retainer 216 of the second fuel retainer assembly 212B. By enabling each heater 230A, 230B to selectively and individually activated, the user is better able to create any desired scent or combination of scents from the warmer assembly 210. Alternatively, the warmer assembly 210 can be designed with only a single heater, which will selectively and simultaneously heat each of the retainer supporters 254A, 254B.

Further, as illustrated in FIG. 2A, the warmer 214 again includes the air mover 232, e.g., a fan, to selectively help to better move and circulate the air within and around the warmer body 226 to more effectively circulate the scents, aromas or fragrances that emanate from the retainer assemblies 212A, 212B upon the melting of the meltable fuel 218 when the retainer assemblies 212A, 212B are heated.

The design and/or positioning of the air mover 232 can be varied. As illustrated, the air mover 232 can include a fan that is positioned substantially horizontally below and/or parallel to the retainer supporters 254A, 254B. Moreover, in certain embodiments, the air mover 232 is coupled to the retainer support assembly 228. For example, in the embodiment illustrated in FIG. 2A, the air mover 232 is positioned and/or coupled about the support shaft 256. With this design, when the air mover 232 is activated by the power source 234, the air mover 232 can rotate with (e.g., the support shaft 256 itself can be rotated) and/or about the support shaft 256 to move air within the warmer body 226 in a generally upward direction toward the retainer supporters 254A, 254B. Additionally, the air mover 232 can direct air through the support vents 262 that are provided within each of the retainer supporters 254A, 254B to enable better air flow, and, thus, to enable better fragrance throw (circulation) upon the melting of the meltable fuel 218 within the fuel retainer 216 of each of the retainer assemblies 212A, 212B. Alternatively, the air mover 232 can have a different design and/or be positioned in a different manner. Still alternatively, the warmer assembly 210 can include a separate and individual air mover 232 for each of the retainer supporters 254A, 254B. For example, in an embodiment in which the retainer support assembly 228 includes two support shafts 256, a separate and individual air mover 232 can be positioned and/or coupled about each of the support shafts 256.

The power source 234 selectively provides power to the first heater 230A, the second heater 230B and the air mover 232. More particularly, the power source 234 can selectively activate the first heater 230A, e.g., between an on and off setting and/or between varying heat levels; the power source 234 can selectively activate the second heater 2308, e.g., between an on and off setting and/or between varying heat levels; and the power source 234 can selectively activate the air mover 232, e.g., between an on and off setting and/or between varying speed settings. Additionally, in certain embodiments, the power source 234 can include a tinier mechanism that allows the heaters 230A, 230B and/or the air mover 232 to be selectively activated for any desired lengths of time. Moreover, as noted above, since each of the heaters 230A, 230B can be selectively activated by the power source 234, the user is better able to create any desired scents or combinations of scents, as desired.

FIG. 2B is a simplified partially exploded front view illustration of the warmer assembly 210 of FIG. 2A. More particularly, FIG. 2B illustrates the first fuel retainer assembly 212A and the second fuel retainer assembly 212B each positioned above the warmer 214 and ready to be selectively and individually positioned within the warmer body 226.

FIG. 2C is a simplified top view illustration of a portion of the warmer assembly 210 of FIG. 2A. More particularly, FIG. 2C is a simplified top view illustration of the warmer 214, with the first fuel retainer assembly 212A and the second fuel retainer assembly 212B having been removed for purposes of clarity. As shown in FIG. 2C, the warmer body 226 can again have a cross-section that is substantially oval-shaped. Additionally, each of the first retainer supporter 254A and the second retainer supporter 254B can also be substantially oval-shaped so as to more effectively receive and retain the first fuel retainer assembly 212A (illustrated in FIG. 2A) and the second fuel retainer assembly 212B (illustrated in FIG. 2A). Moreover, each of the retainer supporters 254A, 254B can be oriented to enable the retainer supporters 254A, 254B to fit more compactly within the warmer body 226. In particular, the orientation of the substantially oval-shaped retainer supporters 254A, 254B can be substantially perpendicular to the orientation of the substantially oval-shaped warmer body 226. Alternatively, the warmer body 226 can have a cross-section that is other than substantially oval-shaped, the retainer supporters 254A, 254B can be other than substantially oval-shaped, and/or the retainer supporters 254A, 254B can be positioned and/or oriented in a different manner within the warmer body 226. For example, in certain non-exclusive, alternative embodiments, the cross-section of the warmer body 226 and/or the retainer supporters 254A, 254B can be substantially circle-shaped, square-shaped, rectangle-shaped, hexagon-shaped, octagon-shaped, or some other shape.

Additionally, FIG. 2C further illustrates the one or more support vents 262 that can be incorporated into the design of each of the retainer supporters 254A, 254B. As provided above, the one or more support vents 262 are designed to provide and/or enable better air flow, e.g., from the operation of the air mover 232 (illustrated in FIG. 2A), and, thus, better fragrance throw (circulation) upon the melting of the meltable fuel 218 (illustrated in FIG. 2A). Further, FIG. 2C also illustrates a portion of the plate coupler 268 that extends substantially between and couples the first retainer supporter 254A and the second retainer supporter 254B.

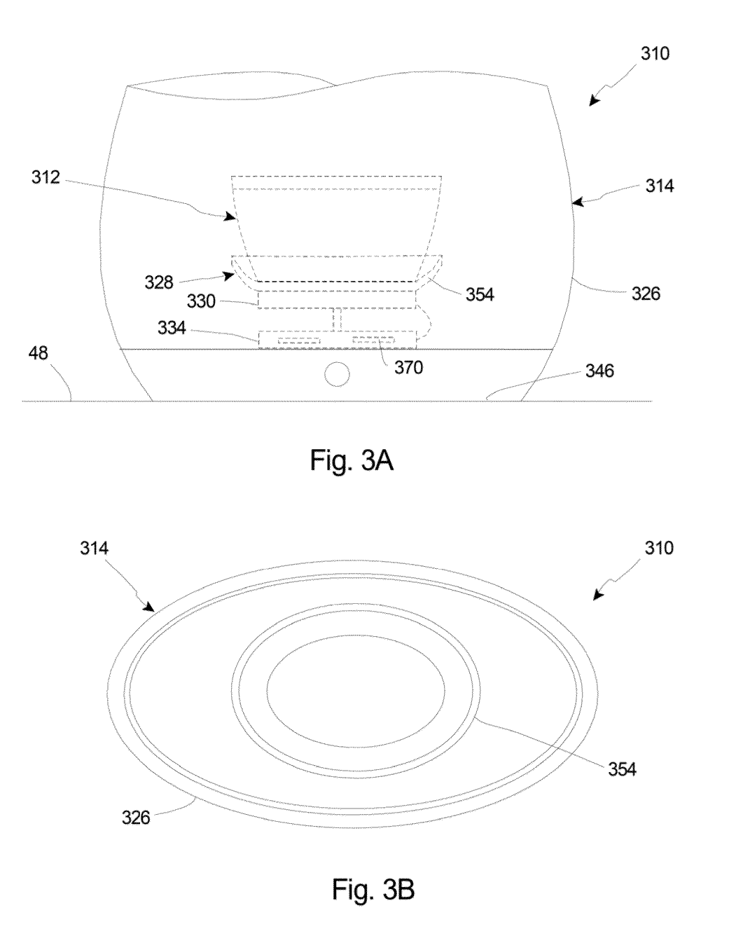

FIG. 3A is a simplified front view illustration of still another embodiment of the warmer assembly 310. The design of the warmer assembly 310 is somewhat similar to the warmer assembly 10 illustrated and described above in relation to FIGS. 1A-1D. For example, in this embodiment, the warmer assembly 310 includes a fuel retainer assembly 312 (illustrated in phantom) and a warmer 314 that are somewhat similar in design and shape to the fuel retainer assembly 12 and the warmer 14 illustrated and described above in relation to FIGS. 1A-1D.

However, the warmer 314 includes certain differences as compared to the warmer 14 illustrated and described above. In particular, in this embodiment, the warmer 314 includes fewer features than the warmer 14 illustrated and described above. For example, in this embodiment, the warmer 314 does not include an air mover. Additionally, in the absence of an air mover, the retainer supporter 354 (illustrated in phantom) of the retainer support assembly 328 (illustrated in phantom) may not include any support vents. Further, as illustrated, the warmer body 326 may not include any base supports, and the base 346 of the warmer body 326 can rest directly upon the surface 48.

Additionally, as illustrated in FIG. 3A, the power source 334 (illustrated in phantom) can include one or more batteries 370 (illustrated in phantom) that can be used to selectively provide power to the heater 330 (illustrated in phantom). Although not specifically illustrated in FIG. 3A, the power source 334 can include a switch and/or a timing mechanism that can selectively activate the heater 330, e.g., between an on and off setting and/or between varying heat levels, and/or selectively activate the heater 330 for any desired lengths of time.

FIG. 3B is a simplified top view illustration of the warmer assembly 310 of FIG. 3A. More particularly, FIG. 3B is a simplified top view illustration of the warmer 314, with the fuel retainer assembly 312 having been removed for purposes of clarity. As shown in FIG. 3B, the warmer body 326 can have a cross-section that is substantially oval-shaped to match the substantially oval-shaped cross-section of the fuel retainer assembly 312 (illustrated in FIG. 3A). Additionally, the retainer supporter 354 can also be substantially oval-shaped so as to more effectively receive and retain the fuel retainer assembly 312. Alternatively, the warmer body 326 can have a cross-section that is other than substantially oval-shaped, and/or the retainer supporter 354 can be other than substantially oval-shaped. For example, in certain non-exclusive, alternative embodiments, the cross-section of the warmer body 326 and/or the retainer supporter 354 can be substantially circle-shaped, square-shaped, rectangle-shaped, hexagon-shaped, octagon-shaped, or some other shape.

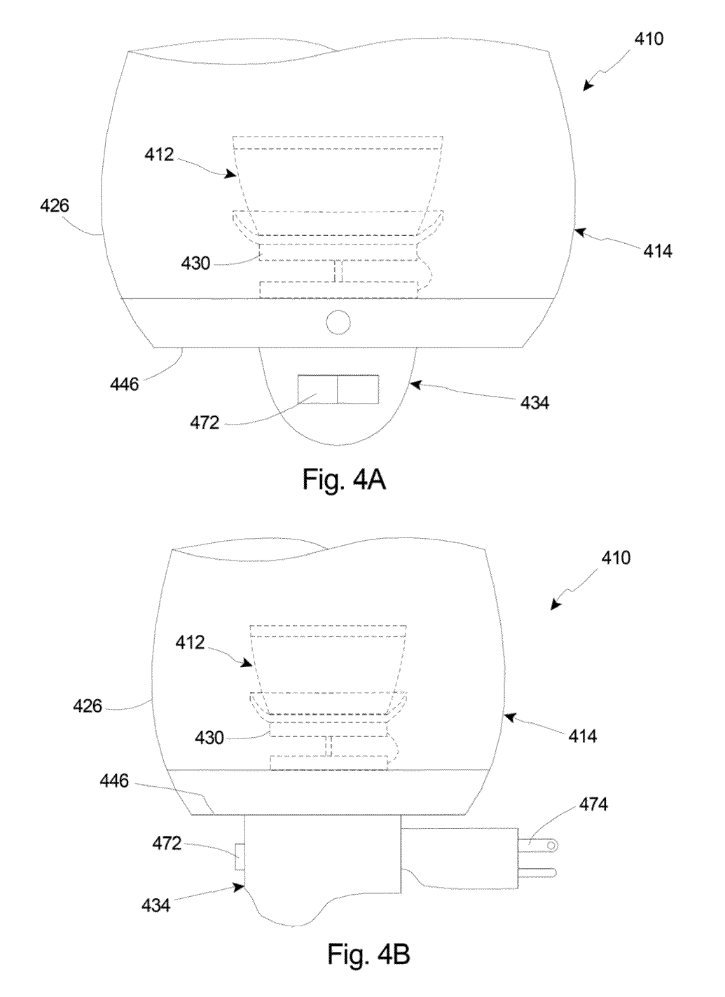

FIG. 4A is a simplified front view illustration of still yet another embodiment of the warmer assembly 410. Additionally, FIG. 4B is a simplified side view illustration of the warmer assembly 410 illustrated in FIG. 4A. The design of the warmer assembly 410 is substantially similar to the warmer assembly 310 illustrated and described above in relation to FIGS. 3A-3B. For example, in this embodiment, the warmer assembly 410 includes a fuel retainer assembly 412 (illustrated in phantom) and a warmer 414 that are substantially similar in design and shape to the fuel retainer assembly 312 and the warmer 314 illustrated and described above in relation to FIGS. 3A-3B.

However, in this embodiment, the power source 434 (illustrated partially in phantom) has a different design. In particular, in this embodiment, the power source 434 includes a switch 472 that selectively activates the power that can be supplied to the heater 430 (illustrated in phantom) and a plug 474 that enables the warmer 414 and, thus, the warmer body 426 to be directed plugged into an outlet (not illustrated). As shown, the switch 472 and the plug 474 can be coupled substantially adjacent to the base 446 of the warmer body 426. Alternatively, the switch 472 and/or the plug 474 can be coupled to the warmer body 426 in a different location.

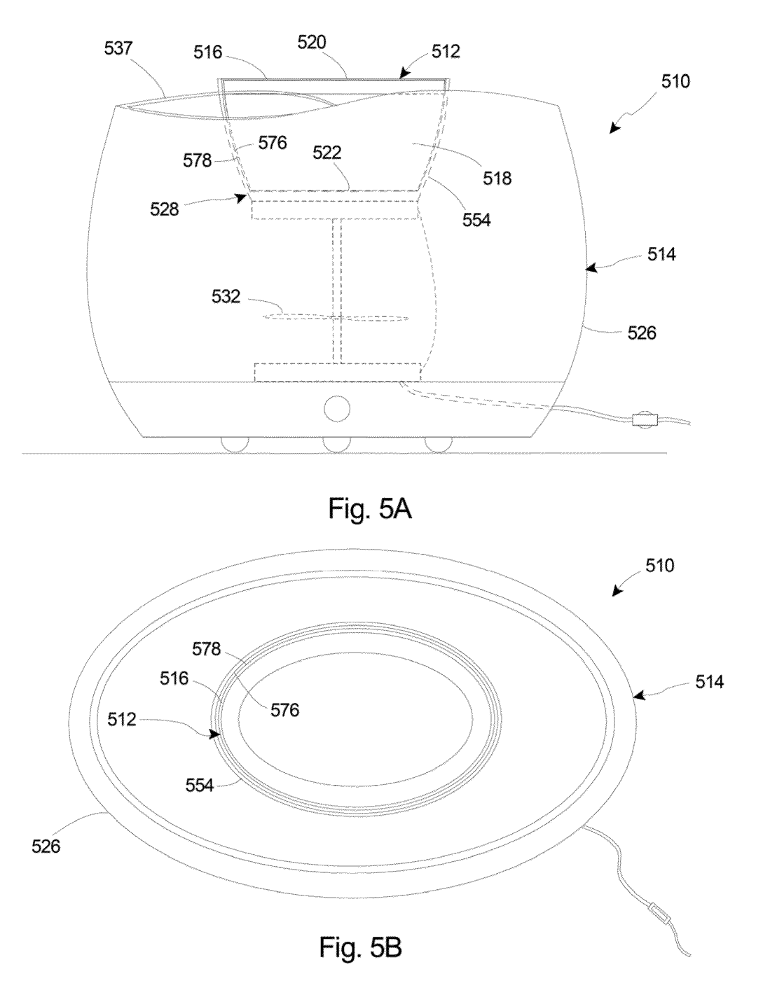

FIG. 5A is a simplified front view of still yet another embodiment of the warmer assembly 510. The warmer assembly 510 is somewhat similar to the warmer assembly 10 illustrated and described above in relation to FIGS. 1A-1D. For example, in this embodiment, the warmer assembly 510 again includes a fuel retainer assembly 512 (illustrated partially in phantom), and a warmer 514 that selectively receives and retains the fuel retainer assembly 512.

However, as illustrated in FIG. 5A, the fuel retainer assembly 512 again includes a fuel retainer 516 (illustrated partially in phantom) and a meltable fuel 518 (illustrated partially in phantom), e.g., a candle, scented wax, or other appropriate meltable fuel, which is retained by the fuel retainer 516. Additionally, in this embodiment, when the fuel retainer assembly 512 is selectively retained by the retainer support assembly 528 within the warmer body 526, the fuel retainer assembly 512 extends slightly above the top edge 537 of the warmer body. For example, in certain embodiments, the fuel retainer assembly 512 can extend above the top edge 537 of the warmer body 526 by between approximately 0.10 and 0.50 inches. In particular, in one such embodiment, when the fuel retainer assembly 512 is selectively retained by the retainer support assembly 528 the fuel retainer assembly 512 can extend above the top edge 537 of the warmer body 526 by approximately 0.25 inches. Alternatively, when the fuel retainer assembly 512 is selectively retained by the retainer support assembly 528 the fuel retainer assembly 512 can extend above the top edge 537 of the warmer body 526 by greater than 0.50 inches or less than 0.10 inches.

Additionally, the fuel retainer 516 can have a cross-section that is substantially oval-shaped and the fuel retainer 516 can be tapered somewhat such that the retainer top 520 of the fuel retainer 516 is somewhat larger and/or wider than the retainer bottom 522 (illustrated in phantom) of the fuel retainer 516. Further, in this embodiment, the retainer supporter 554 of the retainer support assembly 528 can have an inner support surface 576 with a shape that is substantially similar to the shape of an outer surface 578 of the fuel retainer 516. Stated in another manner, substantially the entirety of the inner support surface 576 of the retainer supporter 554 can be substantially directly adjacent to the outer surface 578 of the fuel retainer 516, and/or substantially the entirety of the outer surface 578 of the fuel retainer 516 can be substantially directly adjacent to the inner support surface 576 of the retainer supporter 554. With this design, the air mover 532 can effectively disperse any scent, aroma, and/or fragrance from the meltable fuel 518 without the need for forming vents within the retainer supporter 554, as the air mover 532 will be moving the sir substantially close to the meltable fuel 518 as it is melted within the fuel retainer 516.

FIG. 5B is a simplified top view illustration of the warmer assembly 510 illustrated in FIG. 5A. More particularly, FIG. 5B is a simplified top view illustration of the warmer 514 and the fuel retainer assembly 512 positioned substantially within the warmer body 526 of the warmer 514. As shown in FIG. 5B, the warmer body 526 can have a cross-section that is substantially oval-shaped to match the substantially oval-shaped cross-section of the fuel retainer 516. Additionally, the retainer supporter 554 can also be substantially oval-shaped so as to more effectively receive and retain the fuel retainer assembly 512. Moreover, FIG. 5B again illustrates that the shape of the retainer supporter 554 so closely resembles and/or matches the shape of the fuel retainer 516 that the inner support surface 576 of the retainer supporter 554 is substantially directly adjacent to the outer surface 578 of the fuel retainer 516. Alternatively, the warmer body 526, the retainer supporter 554 and/or the fuel retainer 516 can have a cross-section that is other than substantially oval-shaped. For example, in certain non-exclusive, alternative embodiments, the cross-section of the warmer body 526, the retainer supporter 554 and/or the fuel retainer 516 can be substantially circle-shaped, square-shaped, rectangle-shaped, hexagon-shaped, octagon-shaped, or some other shape.

While a number of exemplary aspects and embodiments of a warmer 10 have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A warmer for use with a fuel retainer assembly of a warmer assembly, the warmer comprising:
a retainer support assembly that is adapted to support the fuel retainer assembly;
a heater that selectively heats the retainer support assembly; and
an air mover that is coupled to the retainer support assembly, wherein the air mover moves air substantially adjacent to the retainer support assembly.

2. The warmer of claim 1 wherein the retainer support assembly includes a retainer supporter that is adapted to retain the fuel retainer assembly.

3. The warmer of claim 2 wherein the retainer supporter includes one or more support vents, and wherein activation of the air mover directs air through the support vents.

4. The warmer of claim 2 wherein the retainer support assembly further includes a support shaft that supports the retainer supporter.

5. The warmer of claim 4 wherein the air mover is positioned about the support shaft.

6. The warmer of claim 1 further comprising a warmer body, wherein the retainer support assembly, the air mover and the heater are positioned substantially within the warmer body.

7. The warmer of claim 1 wherein the retainer support assembly includes a first retainer supporter that is adapted to retain the fuel retainer assembly, and a second retainer supporter that is adapted to simultaneously retain a second fuel retainer assembly.

8. A warmer assembly including a fuel retainer assembly having a meltable fuel and a fuel retainer that retains the meltable fuel, and the warmer of claim 1 that selectively retains the fuel retainer assembly, wherein the heater selectively provides heat the retainer support assembly to melt the meltable fuel.

9. The warmer assembly of claim 8 wherein the retainer support assembly includes a retainer supporter that is adapted to retain the fuel retainer assembly, the retainer supporter having an inner support surface having a first shape, and wherein the fuel retainer has an outer surface having a second shape, the second shape being substantially similar to the first shape.

10. A warmer for use with a first fuel retainer assembly and a second fuel retainer assembly, the warmer comprising:
a retainer support assembly that is adapted to simultaneously retain the first fuel retainer assembly and the second fuel retainer assembly; and
a heater assembly that selectively provides heat to the retainer support assembly.

11. The warmer of claim 10 further comprising an air mover that is coupled to the retainer support assembly, wherein activation of the air mover moves the air substantially adjacent to the retainer support assembly.

12. The warmer of claim 10 wherein the retainer support assembly includes a first retainer supporter that is adapted to retain the first fuel retainer assembly, and a second retainer supporter that is adapted to simultaneously retain the second fuel retainer assembly.

13. The warmer of claim 12 wherein the retainer support assembly further includes a support shaft that supports the first retainer supporter and the second retainer supporter.

14. The warmer of claim 13 further comprising an air mover that is positioned about the support shaft, wherein activation of the air mover moves the air substantially adjacent to the first retainer supporter and the second retainer supporter.

15. The warmer of claim 12 wherein at least one of the first retainer supporter and the second retainer supporter includes one or more support vents, and further comprising an air mover that is coupled to the retainer support assembly, wherein activation of the air mover directs air through the support vents.

16. The warmer of claim 12 wherein the heater assembly includes a first heater that selectively provides heat to the first retainer supporter, and a second heater that selectively provides heat to the second retainer supporter.

17. The warmer of claim 10 further comprising a warmer body, wherein the retainer support assembly and the heater are positioned substantially within the warmer body.

18. A warmer assembly including (i) a first fuel retainer assembly having a first meltable fuel and a first fuel retainer that retains the first meltable, fuel; (ii) a second fuel retainer assembly having a second meltable fuel and a second fuel retainer that retains the second meltable fuel; and (iii) the warmer of claim 11 that selectively and simultaneously retains the first fuel retainer assembly and the second fuel retainer assembly, wherein the heater assembly includes a first heater that selectively provides heat the retainer support assembly to melt the first meltable fuel, and a second heater that selectively provides heat the retainer support assembly to melt the second meltable fuel.

19. A warmer for use with a first fuel retainer assembly, the warmer comprising:
a retainer support assembly including (i) a first retainer supporter that is adapted to retain the first fuel retainer assembly, the first retainer supporter including one or more first support vents, and (ii) a support shaft that supports the first retainer supporter;
a first heater that selectively provides heat to the first retainer supporter;
an air mover that is positioned about the support shaft, wherein activation of the air mover directs air through the first support vents;
a warmer body, wherein the retainer support assembly, the first heater and the air mover are positioned substantially within the warmer body; and
a power source that selectively activates the first heater and the air mover.

20. The warmer of claim 19 wherein the retainer support assembly further includes a second retainer supporter that is adapted to simultaneously retain a second fuel retainer assembly, the second retainer supporter including one or more second support vents, and the support shaft supporting the second retainer supporter; and further comprising a second heater that selectively provides heat to the second retainer supporter, wherein activation of the air mover directs air through the second support vents, and wherein the power source selectively activates the second heater.
